(19) United States
(12) Patent Application Publication
(10) Pub. No.: US 2021/0231653 A1
(43) Pub. Date: Jul. 29, 2021

(54) MICROFLUIDIC CHIP, LIQUID SAMPLE DETECTION DEVICE AND METHOD

(71) Applicant: BOE TECHNOLOGY GROUP CO., LTD., Beijing (CN)

(72) Inventors: Hao TANG, Beijing (CN); Quanguo ZHOU, Beijing (CN); Qin LIU, Beijing (CN); Jiuyang CHENG, Beijing (CN); Zhidong WANG, Beijing (CN); Lijia ZHOU, Beijing (CN); Yancheng LU, Beijing (CN); Ronghua LAN, Beijing (CN)

(21) Appl. No.: 16/964,449

(22) PCT Filed: Feb. 24, 2020
(86) PCT No.: PCT/CN2020/076381

§ 371 Date: Jul. 23, 2020

(30) Foreign Application Priority Data
Apr. 2, 2019 (CN) 201910261840.6

(51) Int. Cl. G01N 33/543; B01L 3/00; G01N 27/74

(52) U.S. Cl. CPC G01N 33/54373; B01L 3/502715; B01L 2200/027; G01N 27/745; B01L 2300/06; G01N 33/54326

(57) ABSTRACT

A microfluidic chip, a liquid sample detection device and a liquid sample detection method are provided. The microfluidic chip includes: a liquid inlet; a waste tank; an accommodating chamber respectively communicated with the liquid inlet and the waste tank at two ends of the accommodating chamber; and at least one giant magnetoresistance structure attached to a wall of the accommodating chamber and having a marker attached thereto. The giant magnetoresistance structure is configured to be attached with magnetic bead particles combined with a to-be-detected object in a liquid sample through a combination of the to-be-detected object and the marker.

### CROSS-REFERENCE TO RELATED APPLICATIONS

This is a National Phase Application filed under 35 U.S.C. 371 as a national stage of PCT/CN2020/076381, filed on Feb. 24, 2020, an application claiming the benefit of Chinese Patent Application No. 201910261840.6, filed with the SIPO on Apr. 2, 2019, the content of each of which is hereby incorporated by reference in its entirety.

### TECHNICAL FIELD

The present disclosure belongs to the technical field of sample detection, and in particular, to a microfluidic chip, a liquid sample detection device and a liquid sample detection method.

### BACKGROUND

Vaccines (e.g. antigenic vaccines) are made of inactivated or attenuated viruses or bacteria, and are injected into bodies to make an immune system of the bodies to generate an immunity against a corresponding disease, so as to prevent the disease in advance. In general, the storage conditions of the vaccines are strict, and inactivated vaccines usually need to be stored at 2□-8□, and will become ineffective if the temperature is too high or too low. In order to ensure the effectiveness of the vaccines, it is necessary to detect the vaccines before inoculation of human bodies with the vaccines.

### SUMMARY

In one aspect, a microfluidic chip is provided. The microfluidic chip includes a liquid inlet; a waste tank; an accommodating chamber respectively communicated with the liquid inlet and the waste tank at two ends of the accommodating chamber; and at least one giant magnetoresistance structure attached to a wall of the accommodating chamber and having a marker attached thereto. The giant magnetoresistance structure is configured to be attached with magnetic bead particles combined with a to-be-detected object in a liquid sample through a combination of the to-be-detected object and the marker.

In one embodiment, the giant magnetoresistance structure includes a resistance unit made of a giant magnetoresistance material, and resistance of the resistance unit is decreased under the function of a magnetic field.

In one embodiment, the microfluidic chip includes a plurality of giant magnetoresistance structures arranged in a column along the accommodating chamber.

In one embodiment, a same type of marker is attached to the plurality of giant magnetoresistance structures.

In one embodiment, different types of markers are attached to the plurality of giant magnetoresistance structures.

In one embodiment, the resistance unit is formed by winding a fine wire made of a nickel-iron-chromium-cobalt material.

In one embodiment, the magnetic bead particles are disposed at the liquid inlet.

In one embodiment, the giant magnetoresistance structure further includes a heating unit configured to heat the marker on the giant magnetoresistance structure, so as to promote the combination of the to-be-detected object in the liquid sample and the marker.

In one embodiment, the giant magnetoresistance structure further includes an insulation layer located on a side of the resistance units distal to the heating unit, and the marker are attached to a surface of the insulation layer distal to the resistance units.

In another aspect, a device for detecting a liquid sample is provided. The device includes the above microfluidic chip; an electromagnetic induction array configured to apply a first magnetic field to the giant magnetoresistance structure of the microfluidic chip; and a detection unit configured to detect resistance of the giant magnetoresistance structure under the function of the first magnetic field, so as to obtain a number of the magnetic bead particles attached to the giant magnetoresistance structure and determine a content of the to-be-detected object in the liquid sample.

In one embodiment, the electromagnetic induction array is further configured to apply the first magnetic field to the giant magnetoresistance structure for activating the magnetic bead particles attached to the giant magnetoresistance structure to generate a second magnetic field, so that resistance of a resistance unit, which is made of a giant magnetoresistance material, of the giant magnetoresistance structure is decreased to a first resistance value. The detection unit is further configured to detect the first resistance value of the resistance unit, and calculate a difference between the first resistance value and a reference value. The reference value is a resistance value of the resistance unit of the giant magnetoresistance structure when the first magnetic field is applied to the giant magnetoresistance structure in the case that no magnetic bead particle is attached to the giant magnetoresistance structure.

In one embodiment, the giant magnetoresistance structure includes the resistance unit located in a first plane, and directions of at least part of magnetic field lines of the first magnetic field and the second magnetic field are parallel to the first plane at the resistance unit.

In one embodiment, the electromagnetic induction array is further configured to generate a third magnetic field for driving the liquid sample to flow to be in contact with the giant magnetoresistance structure and driving the liquid sample to flow away from the giant magnetoresistance structure.

In one embodiment, the liquid sample is a vaccine, the to-be-detected object is an antigen in the vaccine, and the marker is an antibody corresponding to the antigen.

In another aspect, a method for detecting a liquid sample is provided. The method includes: mixing a liquid sample with magnetic bead particles to combine a to-be-detected object in the liquid sample be combined with the magnetic bead particles; causing the liquid sample to be in contact with a giant magnetoresistance structure to which a marker is attached, such that as to fix the magnetic bead particles are attached to the giant magnetoresistance structure through a combination of the to-be-detected object and the marker; applying a first magnetic field to the giant magnetoresistance structure; and detecting resistance of the giant magnetoresistance structure under the function of the first magnetic field, so as to obtain the a number of the magnetic bead particles attached to the giant magnetoresistance structure and determine a content of the to-be-detected object in the liquid sample.

In one embodiment, applying the first magnetic field to the giant magnetoresistance structure includes: applying the first magnetic field to the giant magnetoresistance structure for activating the magnetic bead particles attached to the giant magnetoresistance structure to generate a second magnetic field, so that resistance of a resistance unit, which is made of a giant magnetoresistance material, of the giant magnetoresistance structure is decreased to a first resistance value. The step of detecting the resistance of the giant magnetoresistance structure under the function of the first magnetic field includes: detecting the first resistance value of the resistance unit, and calculating a difference between the first resistance value and a reference value. The reference value is a resistance value of the resistance unit of the giant magnetoresistance structure when the first magnetic field is applied to the giant magnetoresistance structure in the case that no magnetic bead particle is attached to the giant magnetoresistance structure.

In one embodiment, the step of causing the liquid sample be in contact with the giant magnetoresistance structure to which the marker is attached includes: generating a third magnetic field for driving the liquid sample to flow to be in contact with the giant magnetoresistance structure and driving the liquid sample to flow away from the giant magnetoresistance structure.

In one embodiment, the method further includes: heating the marker attached to the giant magnetoresistance structure to promote the combination of the to-be-detected object in the liquid sample and the marker.

In one embodiment, the liquid sample is a vaccine, the to-be-detected object is an antigen in the vaccine, and the marker is an antibody corresponding to the antigen.

### BRIEF DESCRIPTION OF THE DRAWINGS

FIG. 1 is a flowchart illustrating a method for detecting a liquid sample according to an embodiment of the present disclosure;

FIG. 2 is a sectional view of a device for detecting a liquid sample according to an embodiment of the present disclosure;

FIG. 3 is a top view of a microfluidic chip according to an embodiment of the present disclosure;

FIG. 4 is a schematic diagram of a resistance unit according to an embodiment of the present disclosure; and

FIG. 5 is a schematic diagram illustrating a state of magnetic bead particles in a liquid sample attached to a giant magnetoresistance structure having marker.

### DETAILED DESCRIPTION

In order to enable those skilled in the art to better understand the technical solutions of the present disclosure, a microfluidic chip, a liquid sample detection device and method provided by the present disclosure are described in detail below with reference to the accompanying drawings.

On the one hand, recently since a sample used for vaccine detection requires such a high dose that the remaining dose of detected vaccine cannot meet the requirement of vaccination, the existing vaccine detection methods can only make spot check on a batch of vaccines and cannot determine whether each of the vaccines is effective; on the other hand, the existing vaccine detection methods have the problems of long detection time, expensive equipment, and complicated operations.

An embodiment of the present disclosure provides a method for detecting a liquid sample, including steps S21, S22, and S23.

At step S21, a liquid sample is mixed with a plurality of magnetic bead particles, so as to allow a to-be-detected object in the liquid sample to be combined with the magnetic bead particles.

When the liquid sample comes into contact with the plurality of magnetic bead particles, the to-be-detected object may be combined with the magnetic bead particles due to its own properties. Specifically, the to-be-detected object may be combined with specific magnetic bead particles due to a molecular structure and the specific group of the to-be-detected object.

At step S22, the liquid sample is in contact to a giant magnetoresistance structure with marker attached thereon, so as to attach the magnetic bead particles to the giant magnetoresistance structure through combination of the to-be-detected object and the marker. The giant magnetoresistance structure includes resistance unit made of a giant magnetoresistance material.

The marker is attached to the giant magnetoresistance structure. When the liquid sample is brough into contact with the marker on the giant magnetoresistance structure, the to-be-detected object can be combined with the marker due to its own properties, so that the to-be-detected object that is combined with the magnetic bead particles is attached to the giant magnetoresistance structure, and in turn the magnetic bead particles are attached to the giant magnetoresistance structure.

The resistance unit of the giant magnetoresistance structure is made of a giant magnetoresistance material. The resistance of the resistance unit may obviously decrease when a magnetic field exists around the resistance unit.

At step S23, a first magnetic field is applied to the giant magnetoresistance structure, and the resistance of the giant magnetoresistance structure is detected, so as to obtain the number of the magnetic bead particles attached to the giant magnetoresistance structure, and determine a content of the to-be-detected object in the liquid sample.

When the magnetic bead particles are attached to the giant magnetoresistance structure, the magnetic bead particles may change the resistance of the resistance unit. The larger number of the magnetic bead particles attached to the giant magnetoresistance structure is, the more obvious the influence of the magnetic bead particles on the resistance of the resistance unit is. By detecting a change of the resistance of the resistance unit, the number of the magnetic bead particles can be obtained, and thus the content of the to-be-detected object in the liquid sample can be obtain.

The method for detecting a liquid sample according to the embodiment is mainly used for detecting the content of the to-be-detected object in the liquid sample. The method requires a relatively small amount of liquid sample with simplify operation.

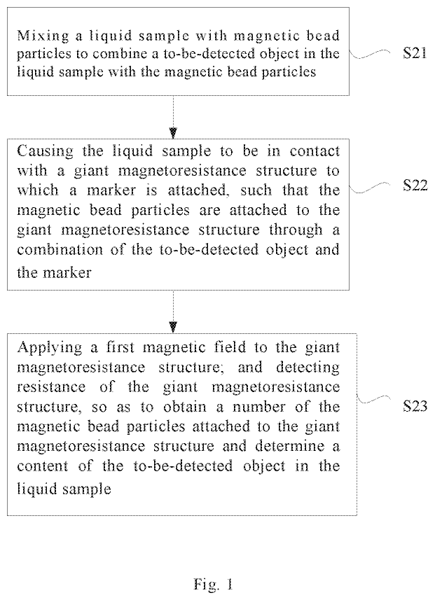

As shown in FIG. 1, at step S21, the liquid sample is mixed with the plurality of magnetic bead particles, so as to allow the to-be-detected object in the liquid sample to be combined with the magnetic bead particles.

When the liquid sample comes into contact with the plurality of magnetic bead particles, the to-be-detected object may be combined with the magnetic bead particles due to its own properties. Specifically, the to-be-detected object may be combined with the specific magnetic bead particles due to the molecular structure and the specific group of the to-be-detected object.

At step S22, the liquid sample is brought into contact with the giant magnetoresistance structure to which the marker is attached, the magnetic bead particles are attached to the giant magnetoresistance structure through the combination of the to-be-detected object and the marker, and the giant magnetoresistance structure includes the resistance unit made of a giant magnetoresistance material.

The marker is attached to the giant magnetoresistance structure; when the liquid sample is brough into contact with the marker on the giant magnetoresistance structure, the to-be-detected object may be combined with the marker due to its own properties, which enables the to-be-detected object that is combined with the magnetic bead particles to be attached to the giant magnetoresistance structure, and further enables the magnetic bead particles to be attached to the giant magnetoresistance structure.

The resistance unit of the giant magnetoresistance structure is made of a giant magnetoresistance material, and the resistance of the resistance unit may be obviously decreased when a magnetic field appears around the resistance unit.

Specifically, the step of making the liquid sample to be in contact with the giant magnetoresistance structure to which the marker is attached includes:

S221, driving the liquid sample to flow to be in contact with the giant magnetoresistance structure; and

S222, driving the liquid sample to flow away from the giant magnetoresistance structure.

The liquid sample is driven in two ways as being driven by an electric field and being driven by a magnetic field.

In the case of being driven by an electric field, a tiny amount of liquid sample (e.g. a droplet of liquid sample) moves because the surface tension thereof is changed under the function of the electric field, so that the droplet of liquid sample may pass through the giant magnetoresistance structure to allow the to-be-detected object to be combined with the marker.

In the case of being driven by a magnetic field, the magnetic field is generated along a moving path of the liquid sample, so that the liquid sample, which contains the magnetic bead particles, is driven by the magnetic field to move along the moving path.

S23, applying the first magnetic field to the giant magnetoresistance structure, and detecting the resistance of the giant magnetoresistance structure, so as to obtain the number of the magnetic bead particles attached to the giant magnetoresistance structure and determine the content of the to-be-detected object in the liquid sample.

In one embodiment, the step of applying the first magnetic field to the giant magnetoresistance structure and detecting the resistance of the giant magnetoresistance structure includes:

S231, applying the first magnetic field to the giant magnetoresistance structure to activate the magnetic bead particles attached to the giant magnetoresistance structure to generate a second magnetic field, and the resistance of the resistance unit is decreased to a first resistance value R1 under the function of the first and second magnetic fields; and

S232, detecting the first resistance value R1 of the resistance unit.

The first magnetic field affects both the resistance unit of the giant magnetoresistance structure and the magnetic bead particles on the giant magnetoresistance structure. Specifically, the resistance of the resistance unit is decreased and the second magnetic field is generated by the magnetic bead particles under the function of the first magnetic field, and the resistance of the resistance unit is further decreased to the first resistance value R1 under the function of the second magnetic field. By detecting a reduction amount (Rref-R1) of the resistance of the resistance unit, whether the magnetic bead particles are attached to the giant magnetoresistance structure and the number of the magnetic bead particles attached the giant magnetoresistance structure can be determined, with Rref representing a reference value of the resistance unit. Before the liquid sample is detected, that is, when the giant magnetoresistance structure is not in contact with the liquid sample mixed with the magnetic bead particles, or when no magnetic bead particles are attached to the giant magnetoresistance structure, the first magnetic field is applied to the giant magnetoresistance structure. The resistance unit of the giant magnetoresistance structure have (or resistance value thereof is decreased to) the reference value Rref under the function of the first magnetic field.

After the magnetic bead particles are attached to the giant magnetoresistance structure, the first magnetic field is kept being applied to the giant magnetoresistance structure, so that the magnetic bead particles are excited or activated by the first magnetic field to generate the second magnetic field, and the resistance of the resistance unit of the giant magnetoresistance structure is decreased to the first resistance value R1 under the function of the first and second magnetic fields. A difference between the first resistance value R1 and the reference value Rref is calculated, and the number of the magnetic bead particles on the giant magnetoresistance structure can be determined according to the difference.

The larger number of the magnetic bead particles attached on the giant magnetoresistance structure is, the more obvious the influence of the magnetic bead particles on the resistance of the resistance unit is. By detecting the reduction amount of the resistance of the resistance unit, the number of the magnetic bead particles can be obtained, and thus the content of the to-be-detected object in the liquid sample can be obtained.

A resistance unit 11 of the giant magnetoresistance structure is located in a first plane, and directions, at the resistance unit, of at least part of the magnetic field lines of the first magnetic field and the second magnetic field are parallel to the first plane, as shown by the dotted line in FIG. 5.

Any magnetic field may be decomposed into a vertical magnetic field and a parallel magnetic field. The resistance unit is insensitive to the vertical magnetic field, but is sensitive to the parallel magnetic field. The vertical magnetic field refers to a magnetic field perpendicular to the resistance unit, and the parallel magnetic field refers to a magnetic field parallel to the resistance unit.

Since the magnetic field lines of the first magnetic field and the second magnetic field are parallel to the first plane at the resistance unit 11, the accuracy of the influence of the first magnetic field and the second magnetic field on the resistance of the resistance unit can be ensured, thereby obtaining the accurate number of the magnetic bead particles attached to the giant magnetoresistance structure, and accurately detecting the content of the to-be-detected object in the liquid sample.

The method for detecting a liquid sample according to the embodiment is mainly used for detecting the content of the to-be-detected object in the liquid sample. The method requires a relatively small amount of liquid sample, and is simple and convenient to carry out.

In one embodiment, the liquid sample is a vaccine, the to-be-detected object is an antigen in the vaccine, and the marker is an antibody corresponding to the antigen.

In one embodiment, the magnetic bead particles are biomagnetic beads, and the giant magnetoresistance structure is a giant magnetoresistance biochip. When the vaccine is mixed with the magnetic bead particles, the antigen in the vaccine can be combined with the magnetic bead particles (that is, the magnetic bead-antigen chain is formed); and the antigen can be specifically bound to the antibody on the giant magnetoresistance structure (that is, the magnetic bead-antigen-antibody chain is formed), when the vaccine combined with the magnetic bead particles passes through the giant magnetoresistance structure, so that the magnetic bead particles are attached to the giant magnetoresistance structure.

When the magnetic bead particles are attached to the giant magnetoresistance structure, the magnetic bead particles may change the resistance of the resistance unit, and the larger number of the magnetic bead particles attached to the giant magnetoresistance structure is, the more obvious the influence of the magnetic bead particles on the resistance of the resistance unit is. By detecting the change of the resistance of the resistance unit, the number of the magnetic bead particles can be obtained, and thus a content of the antigen in the vaccine can be obtained.

The method for detecting a liquid sample according to the embodiment may be used for detecting the content of the antigen in the vaccine. On the one hand, the liquid sample detection method requires a relatively small amount of vaccine (such as 10 μL-20 μL) for detection, therefore each vaccine can be detected, and the detected vaccine can still meet vaccination standards, thereby guaranteeing the effectiveness of each vaccine; on the other hand, the liquid sample detection method has the advantages of simple operation, short detection time, and low cost equipment.

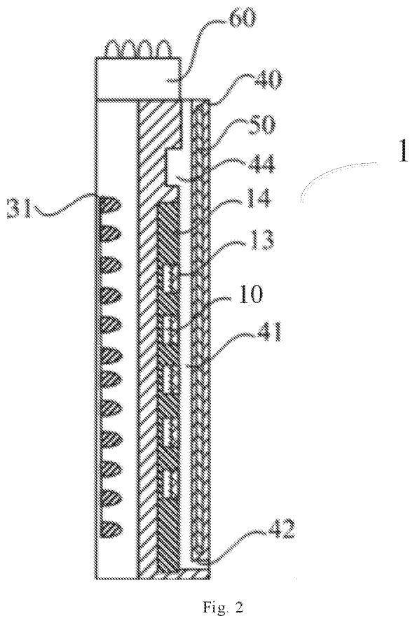

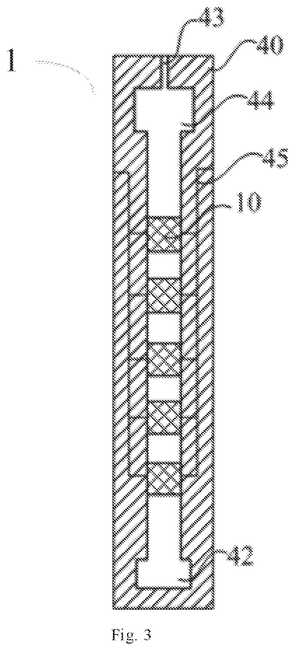

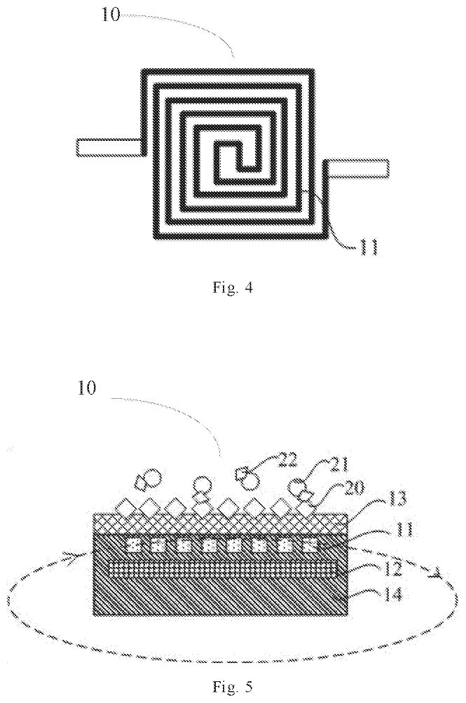

FIG. 2 is a sectional view of a device for detecting a liquid sample according to an embodiment of the present disclosure. FIG. 3 is a top view of a microfluidic chip according to an embodiment of the present disclosure. FIG. 4 is a schematic diagram showing a resistance unit according to an embodiment of the present disclosure. FIG. 5 is a schematic diagram illustrating a state of magnetic bead particles in a liquid sample attached to a giant magnetoresistance structure having marker.

FIG. 4 is a schematic diagram showing a resistance unit according to an embodiment of the present disclosure. FIG. 3 is a top view of a microfluidic chip 1 formed by five (5) giant magnetoresistance structures, but the number of the giant magnetoresistance structures in the microfluidic chip is not limited thereto. FIG. 2 is a sectional view of a device for detecting a liquid sample according to an embodiment of the present disclosure. FIG. 5 is a sectional view illustrating a case in which a giant magnetoresistance structure 10 with the marker 20 attached thereon is in contact with a to-be-detected object 22 in a liquid sample.

As shown in FIG. 1 to FIG. 5, the embodiments of the present disclosure further provide a device for detecting a liquid sample. The device includes a microfluidic chip 1, an electromagnetic induction array 31, and a detection unit 60.

The microfluidic chip 1 includes at least one giant magnetoresistance structure 10. The at least one giant magnetoresistance structure 10 includes a resistance unit 11 made of a giant magnetoresistance material, and has marker 20 attached thereto. The marker 20 can be combined with the to-be-detected object 22 in a liquid sample. A plurality of magnetic bead particles 21 may be mixed with the liquid sample, and in turn may be combined with the to-be-detected object 22 in the liquid sample. When the liquid sample comes into contact with the giant magnetoresistance structure 10, the magnetic bead particles 21 in the liquid sample are attached to the giant magnetoresistance structure 10.

The electromagnetic induction array 31 applies a first magnetic field to the giant magnetoresistance structure of the microfluidic chip. The electromagnetic induction array 31 may be formed by single-point strobed micro-electromagnet units.

The detection unit 60 detects the resistance of the resistance unit 11 of the giant magnetoresistance structure 10 under the function of the first magnetic field to obtain the number of the magnetic bead particles 21 attached to the giant magnetoresistance structure 10, and determine a content of the to-be-detected object 22 in the liquid sample.

The marker 20 is attached to the giant magnetoresistance structure 10. When the liquid sample is in contact with the marker 20 on the giant magnetoresistance structure 10, the to-be-detected object 22 can be combined with the marker 20 due to its own properties, that is, the to-be-detected object 22 combined with the magnetic bead particles 21 is fixed to the giant magnetoresistance structure 10, and in turn the magnetic bead particles 21 are attached to the giant magnetoresistance structure 10.

The resistance unit 11 of the giant magnetoresistance structure 10 is made of a giant magnetoresistance material, and the resistance of the resistance unit 11 may be obviously decreased when a magnetic field appears around the resistance unit 11.

The detection unit 60 detects the resistance of the giant magnetoresistance structure 10 of the microfluidic chip 1 to determine the number of the magnetic bead particles 21. The detection unit 60 includes a structure for detecting the resistance of the giant magnetoresistance structure 10, such as a lead 45 configured to output information about the resistance of the giant magnetoresistance structure 10. Since various known structures can be used for detecting the resistance of the giant magnetoresistance structure 10, the detailed structure is not shown in the drawings.

In one embodiment, the microfluidic chip 1 includes a plurality of giant magnetoresistance structures 10.

Different types of markers 20 may be fixed with different giant magnetoresistance structures 10 respectively. There are various types of markers 20, and different types of markers 20 are attached to different giant magnetoresistance structures 10 respectively. Various types of to-be-detected objects 22 exist in the liquid sample, each type of object to be detected 22 may be combined with one or one type of the markers 20, and the different giant magnetoresistance structures 10 may be combined with corresponding types of markers 20 respectively. As a result, the device for detecting the liquid sample can simultaneously detect the contents of the various types of to-be-detected objects 22 in the liquid sample.

In one embodiment, the plurality of giant magnetoresistance structures 10 are arranged in one column.

In the case where the plurality of giant magnetoresistance structures 10 are arranged in one column, the liquid sample may flow through each of the giant magnetoresistance structures 10 in sequence. Therefore, the different types of to-be-detected objects 22 may be sequentially attached to the giant magnetoresistance structures 10 respectively by driving the liquid sample to move along a straight line.

Such arrangement of the plurality of giant magnetoresistance structures 10 may achieve simple driving of the liquid sample and improve detection efficiency.

In one embodiment, the giant magnetoresistance structure 10 further includes a heating unit 12 configured to heat the marker on the giant magnetoresistance structure, so as to promote the combination of the to-be-detected object in the liquid sample and the marker.

The heating unit 12 may be disposed on a side of the resistance units 11, and may be made of an iron-chromium-aluminum alloy material.

Since some certain to-be-detected objects 22 may be combined with the marker 20 only at certain temperature (i.e., an incubation temperature), the heating unit 12 may ensure that different to-be-detected objects 22 may be combined with the corresponding markers 20 very well.

In one embodiment, the giant magnetoresistance structure 10 further includes an insulation layer 13 located on a side of the resistance units 11 distal to the heating unit 12, and the marker 20 is attached to a surface of the insulation layer 13 distal to the resistance unit 11.

The insulation layer 13 separates the resistance units 11 from the marker 20, so as to prevent the properties of the marker 20 from being affected by the resistance units 11 and prevent the combination of the marker 20 and the to-be-detected object 22 from being affected by the resistance units 11. The insulation layer 13 may be made of a polyimide material (i.e., a PI adhesive).

In one embodiment, the electromagnetic induction array 31 applies the first magnetic field to the giant magnetoresistance structure 10, and the magnetic bead particles 21 attached to the giant magnetoresistance structure 10 may be excited by the first magnetic field to generate the second magnetic field. The resistance of the resistance unit 11 may be decreased to the first resistance value R1 under the function of the second magnetic field.

The detection unit 60 detects the first resistance value R1 of the resistance unit 11, and calculates the difference between the first resistance value R1 and the reference value Rref of the resistance.

The resistance unit 11 of the giant magnetoresistance structure 10 is located in the first plane. The directions, at the resistance unit 11, of at least part of the magnetic field lines of the first magnetic field and the second magnetic field are parallel to the first plane. As shown in FIG. 4, the resistance unit 11 may be formed by winding a filament-shaped structure (e.g. a fine wire made of a nickel-iron-chromium-cobalt material) in the first plane.

The first magnetic field affects both the resistance unit 11 of the giant magnetoresistance structure 10 and the magnetic bead particles 21 on the giant magnetoresistance structure 11. Specifically, the resistance of the resistance unit 11 is decreased under the function of the first magnetic field, and the magnetic bead particles 21 are activated by the first magnetic field to generate the second magnetic field. The resistance of the resistance unit 11 is further decreased to the first resistance value R1 under the function of the second magnetic field. The difference, i.e., the reduction amount of the resistance, between the first resistance value R1 and the reference value Rref of the resistance is calculated. By detecting the reduction amount (Rref-R1) of the resistance of the resistance unit 11, whether the magnetic bead particles 21 are attached to the giant magnetoresistance structure 10 and the number of the magnetic bead particles 21 on the giant magnetoresistance structure 10 can be determined, with Rref representing the reference value of the resistance unit.

Before the microfluidic chip is used, that is, when no magnetic bead particle is attached to the giant magnetoresistance structure, the first magnetic field is applied to the giant magnetoresistance structure, and the resistance unit of the giant magnetoresistance structure has (or resistance value thereof is decreased to) the reference value Rref under the function of the first magnetic field.

When the microfluidic chip is used, that is, after the magnetic bead particles are attached to the giant magnetoresistance structure, the first magnetic field is kept being applied to the giant magnetoresistance structure, so that the magnetic bead particles are excited by the first magnetic field to generate the second magnetic field, and the resistance of the resistance unit of the giant magnetoresistance structure is decreased to the first resistance value R1 under the function of the first and second magnetic fields. The difference, i.e., the reduction amount of the resistance, between the first resistance value R1 and the reference value Rref is calculated. The number of the magnetic bead particles on the giant magnetoresistance structure may be determined according to the reduction amount (Rref-R1) of the resistance of the resistance unit. The larger number of the magnetic bead particles 21 attached to the giant magnetoresistance structure 10 is, the more obvious the influence of the magnetic bead particles 21 on the resistance of the resistance unit 11 is. By detecting the reduction amount of the resistance of the resistance unit 11, the number of the magnetic bead particles 21 can be obtained, and in turn the content of the to-be-detected object 22 in the liquid sample can be obtained.

In one embodiment, the microfluidic chip 1 further includes an accommodating structure 40 with an accommodating chamber 41. The plurality of giant magnetoresistance structures 10 are attached to a wall of the accommodating chamber 41 along a length direction of the accommodating chamber 41, and a liquid sample may flow in the accommodating chamber 41.

The liquid sample, when flowing in the accommodating chamber 41 (i.e., a microfluidic channel), may pass through the plurality of giant magnetoresistance structures 10 that are arranged in a column, so that the to-be-detected object 22 in the liquid sample can be combined with the marker 20 on the giant magnetoresistance structures 10, so as to detect the content of the to-be-detected object 22 in the liquid sample.

The accommodating structure 40 may not only simplify the flowing of the liquid sample, but also simplify the contact between the liquid sample and the giant magnetoresistance structures 10, thereby simplifying the structure of the microfluidic chip.

As shown in FIG. 2 and FIG. 3, the accommodating structure 40 includes a liquid inlet 42, an air vent 43, and a waste tank 44. Firstly, the liquid sample to be detected is disposed at the liquid inlet 42. The magnetic bead particles 21 may be arranged at the liquid inlet 42, alternatively the magnetic bead particles 21 may be mixed with the liquid sample in advance outside the accommodating structure 40. And then, the liquid sample is driven to flow along the accommodating chamber 41 to sequentially pass through the plurality of giant magnetoresistance structures 10 on the wall of the accommodating chamber 41, so as to allow the to-be-detected object 22 in the liquid sample to be combined with the marker 20 on the plurality of giant magnetoresistance structures 10. The antigen and the magnetic bead particles, which are not involved in the reaction, continue to move toward the next giant magnetoresistance structure 10. Finally, the remaining liquid is stored in the waste tank 44. The detection unit 60 detects the change of the resistance of the resistance unit 11 of the giant magnetoresistance structures 10 to determine the content of the to-be-detected object 22 in the liquid sample.

The microfluidic chip 1 further includes a fixing member 14 for fixing the giant magnetoresistance structures 10 onto the wall of the accommodation chamber 41.

In one embodiment, the liquid sample detection device further includes a driving device for driving the liquid sample to flow in the accommodating chamber 41. The driving device may be an electric field driving device or a magnetic field driving device.

In the case where an electric field driving device for driving the liquid sample to flow with an electric field, a tiny amount of liquid sample (e.g. a droplet of liquid sample) moves since the surface tension thereof is changed under the function of the electric field, so that the droplet of liquid sample may pass through the giant magnetoresistance structures, and the to-be-detected object may be combined with the marker.

The microfluidic chip requires a tiny amount of sample, for example, 1/10 drop of liquid sample is enough for detection.

When the tiny amount of liquid sample (e.g. a droplet of liquid sample) enters the microfluidic chip 1 via the liquid inlet 42, the electric field driving device may move the droplet of liquid sample by changing the surface tension thereof.

In one embodiment, as shown in FIG. 2, an electric field driving device 50 may include two opposite substrates, electrodes disposed on the substrates, and a circuit configured to apply a voltage to the electrodes. The substrates, the electrodes, and the circuit of the microfluidic structure may be disposed in the accommodating structure 40. Since various structures and arrangement of the substrates, the electrodes and the circuits of the microfluidic structure are well known, the details thereof are omitted from the drawings.

Alternatively, the liquid sample may be driven with a magnetic field. In the case where the magnetic field driving device for driving the liquid sample to flow with a magnetic field, the electric field driving device 50 shown in FIG. 2 may be removed, and the liquid sample is driven only by the electromagnetic induction array 31. Specifically, a magnetic field is generated by the electromagnetic induction array 31 along the accommodating chamber 41. The liquid sample, which contains the magnetic bead particles, is driven by the magnetic field to move along the accommodating chamber 41.

In one embodiment, the detection unit 60 is used for detecting the content of the to-be-detected object 22 in the liquid sample, and the content may indicate a specific content of the to-be-detected object 22 in the liquid sample, and may also indicate whether the content of the to-be-detected object 22 in the liquid sample meets a standard. For example, the detection unit 60 includes an indicator light, a battery, a control chip, and a circuit. The control chip may monitor the reduction amounts of the resistances of the resistance units 11 of the giant magnetoresistance structures 10, and output a corresponding electrical signal to the indicator light. The content information of the to-be-detected object 22 in the liquid sample is finally determined according to the indicator light. For example, when the content of the to-be-detected object 22 in the liquid sample meets the standard, the indicator light is turned on; and when the content of the to-be-detected object 22 in the liquid sample does not meet the standard, the indicator light is not turned on.

The liquid sample is a vaccine, the to-be-detected object 22 is the antigen in the vaccine, and the marker 20 is the antibody corresponding to the antigen.

When the vaccine is mixed with the magnetic bead particles 21, the antigen in the vaccine may be combined with the magnetic bead particles 21 (that is, the magnetic bead-antigen chain is formed). When the vaccine combined with the magnetic bead particles 21 passes through the giant magnetoresistance structure 10, the antigen can be bound to the antibody on the giant magnetoresistance structure 10 (that is, the magnetic bead-antigen-antibody chain is formed) and in turn the magnetic bead particles 21 are attached to the giant magnetoresistance structure 10.

When the magnetic bead particles 21 are attached to the giant magnetoresistance structure 10, the magnetic bead particles 21 may change the resistance of the resistance unit 11. The larger number of the magnetic bead particles 21 attached to the giant magnetoresistance structure 10 is, the more obvious the influence of the magnetic bead particles 21 on the resistance of the resistance unit 11 is. By detecting the change of the resistance of the resistance unit 11, the number of the magnetic bead particles 21 can be obtained, and thus the content of the antigen in the vaccine can be obtained. The magnetic bead particles 21 not only separate antigen, but also play an important role in the process of detecting the content of the antigen.

In addition, since the different antibodies are on the different giant magnetoresistance structures 10 respectively, the device for detecting the liquid sample may perform detection on the polyvalent vaccine.

Alternatively, the same antibody may be attached to the different giant magnetoresistance structures 10, so that the antigen in the vaccine may be fully bound to the antibody on the giant magnetoresistance structures 10, so as to obtain a more accurate content of the antigen in the vaccine.

The device for detecting a liquid sample according to the embodiment may be used for detecting the content of the antigen in the vaccine. On the one hand, a relatively small amount of vaccine is required for detection, therefore each vaccine may be detected without affecting inoculation with the vaccine, thereby guaranteeing the effectiveness of each of the vaccines; on the other hand, the device has the advantages of simple operation, short detection time, and low cost.

It should be understood that the above embodiments are merely exemplary embodiments employed to illustrate the principles of the present disclosure, and the present disclosure is not limited thereto. Without departing from the spirit and essence of the present disclosure, those skilled in the art may make various changes and modifications, and those changes and modifications should be considered to fall within the scope of the present disclosure.

1. A microfluidic chip, comprising:
a liquid inlet;
a waste tank;
an accommodating chamber, respectively communicated with the liquid inlet and the waste tank at two ends of the accommodating chamber; and
at least one giant magnetoresistance structure attached to a wall of the accommodating chamber and having a marker attached thereto, wherein
the giant magnetoresistance structure is configured to be attached with magnetic bead particles combined with a to-be-detected object in a liquid sample through a combination of the to-be-detected object and the marker.

2. The microfluidic chip of claim 1, wherein
each of the giant magnetoresistance structure comprises a resistance unit made of a giant magnetoresistance material, and
resistance of the resistance unit is decreased under the function of a magnetic field.

3. The microfluidic chip of claim 2, wherein
the at least one giant magnetoresistance structure comprises a plurality of giant magnetoresistance structures arranged in a column along the accommodating chamber.

4. The microfluidic chip of claim 3, wherein
a same type of marker is attached to the plurality of giant magnetoresistance structures.

5. The microfluidic chip of claim 3, wherein
different types of markers are attached to the plurality of giant magnetoresistance structures.

6. The microfluidic chip of claim 2, wherein
the resistance unit is formed by winding a fine wire made of a nickel-iron-chromium-cobalt material.

7. The microfluidic chip of claim 6, wherein
the magnetic bead particles are disposed at the liquid inlet.

8. The microfluidic chip of claim 7, wherein the giant magnetoresistance structure further comprises a heating unit configured to heat the marker on the giant magnetoresistance structure, so as to promote the combination of the to-be-detected object in the liquid sample and the marker.

9. The microfluidic chip of claim 8, wherein the giant magnetoresistance structure further comprises an insulation layer located on a side of the resistance units distal to the heating unit, and the marker is attached to a surface of the insulation layer distal to the resistance units.

10. A device for detecting a liquid sample, comprising:
the microfluidic chip of claim 1;
an electromagnetic induction array configured to apply a first magnetic field to the giant magnetoresistance structure of the microfluidic chip; and
a detection unit configured to detect resistance of the giant magnetoresistance structure under the function of the first magnetic field, so as to obtain a number of the magnetic bead particles attached to the giant magnetoresistance structure and determine a content of the to-be-detected object in the liquid sample.

11. The device of claim 10, wherein
the electromagnetic induction array is further configured to apply the first magnetic field to the giant magnetoresistance structure for activating the magnetic bead particles attached to the giant magnetoresistance structure to generate a second magnetic field, so that resistance of a resistance unit, which is made of a giant magnetoresistance material, of the giant magnetoresistance structure is decreased to a first resistance value; and
the detection unit is further configured to detect the first resistance value of the resistance unit, and calculate a difference between the first resistance value and a reference value, wherein the reference value is a resistance value of the resistance unit of the giant magnetoresistance structure when the first magnetic field is applied to the giant magnetoresistance structure in the case that no magnetic bead particle is attached to the giant magnetoresistance structure.

12. The device of claim 11, wherein
the resistance unit is located in a first plane, and
directions, at the resistance unit, of at least part of magnetic field lines of the first magnetic field and the second magnetic field are parallel to the first plane.

13. The device of claim 12, wherein the electromagnetic induction array is further configured to generate a third magnetic field for driving the liquid sample to flow to be in contact with the giant magnetoresistance structure and driving the liquid sample to flow away from the giant magnetoresistance structure.

14. The device of claim 13, wherein the liquid sample is a vaccine, the to-be-detected object is an antigen in the vaccine, and the marker is an antibody corresponding to the antigen.

15. A method for detecting a liquid sample, comprising:
mixing a liquid sample with magnetic bead particles to combine a to-be-detected object in the liquid sample with the magnetic bead particles;
causing the liquid sample to be in contact with a giant magnetoresistance structure to which a marker is attached, such that the magnetic bead particles are attached to the giant magnetoresistance structure through a combination of the to-be-detected object and the marker;
applying a first magnetic field to the giant magnetoresistance structure; and
detecting resistance of the giant magnetoresistance structure under the function of the first magnetic field, so as to obtain a number of the magnetic bead particles attached to the giant magnetoresistance structure and determine a content of the to-be-detected object in the liquid sample.

16. The method of claim 15, wherein
applying the first magnetic field to the giant magnetoresistance structure comprises:
applying the first magnetic field to the giant magnetoresistance structure for activating the magnetic bead particles attached to the giant magnetoresistance structure to generate a second magnetic field, so that resistance of a resistance unit, which is made of a giant magnetoresistance material, of the giant magnetoresistance structure is decreased to a first resistance value, and
detecting the resistance of the giant magnetoresistance structure under the function of the first magnetic field comprises:
detecting the first resistance value of the resistance unit, and calculating a difference between the first resistance value and a reference value, the reference value being a resistance value of the resistance unit of the giant magnetoresistance structure when the first magnetic field is applied to the giant magnetoresistance structure in the case that no magnetic bead particle is attached to the giant magnetoresistance structure.

17. The method of claim 16, wherein
causing the liquid sample to be in contact with the giant magnetoresistance structure to which the marker is attached comprises:
generating a third magnetic field for driving the liquid sample to flow to be in contact with the giant magnetoresistance structure and driving the liquid sample to flow away from the giant magnetoresistance structure.

18. The method of claim 17, further comprising:
heating the marker attached to the giant magnetoresistance structure to promote the combination of the to-be-detected object in the liquid sample and the marker.

19. The method of claim 18, wherein the liquid sample is a vaccine, the to-be-detected object is an antigen in the vaccine, and the marker is an antibody corresponding to the antigen.
